(19) United States
(12) Patent Application Publication
(10) Pub. No.: US 2021/0222182 A1
(43) Pub. Date: Jul. 22, 2021

(54) INDUCED IMMUNOLOGICAL RESPONSE TO CANCEROUS CELLS USING VECTORS CONTAINING VIRAL GENES

(71) Applicant: Walter W. Aller, Tampa Bay, FL (US)

(72) Inventor: Walter W. Aller, Tampa Bay, FL (US)

(21) Appl. No.: 17/151,162

(22) Filed: Jan. 17, 2021

Related U.S. Application Data
(60) Provisional application No. 62/962,921, filed on Jan. 17, 2020.

(51) Int. Cl. C12N 15/79; C07K 14/05; C07K 14/12

(52) U.S. Cl. CPC C12N 15/79; A61K 38/00; C07K 14/12; C07K 14/05

(57) ABSTRACT

A method for treating cancer in mammals using an expression vector to transfect cancer cells in vivo and in situ. The vector includes one or more immunogenic exogenous polypeptides that are expressed by the cancer cells upon transfection. The body's immune response is triggered and directed to attack the transfected cancer cells. Once the immune response to the exogenous peptide on the cancer cells is initiated, an immune response to the other cancer associated or cancer specific antigens on or in the cancer cells takes over and eliminates all cancer cells, transfected or not.

### CROSS-REFERENCE TO RELATED APPLICATIONS

This application claims priority to U.S. Provisional Application Ser. No. 60/962,921 filed on Jan. 17, 2020, the contents of which are hereby incorporated in their entirety.

### STATEMENT REGARDING FEDERALLY SPONSORED RESEARCH OR DEVELOPMENT

Not Applicable.

### NAMES OF PARTIES TO A JOINT RESEARCH AGREEMENT

Not Applicable

### REFERENCE TO SEQUENCE LISTING, A TABLE, OR A COMPUTER PROGRAM LISTING APPENDIX SUBMITTED ON A COMPACT DISC AND INCORPORATION-BY-REFERENCE OF THE MATERIAL

Not Applicable.

### COPYRIGHT NOTICE

Not Applicable

### BACKGROUND OF THE INVENTION

### Field of the Invention

The present invention relates to system, compositions and methods for inducing an immunological response to cancerous cells. More particularly, the invention relates to a vector injected directly into a tumor to induce cancerous cells to express viral and other exogenous immunogenic polypeptides which trigger a response from a cancer patient's own immune system.

### Description of the Related Art

Cancers are a group of diseases involving abnormal cell growth with the potential to invade or spread to other parts of the body. For many cancers the treatment regimen can include one or a combination of surgery, chemotherapy, radiation, bone marrow/stem cell transplants, cancer drugs or immunotherapy. The most common treatments include surgery, chemotherapy, radiation and oral drugs. Chemotherapies utilize drugs that kill tumor cells by intercalation of DNA, inhibition of replication, or prevention of microtubule assembly. Radiotherapy is essentially geared to kill cancer cells by damaging DNA. Although these treatments can be effective there are often many side effects. Furthermore, none of these therapies destroy every single cancer cell. It only takes that one cancer cell to grow back and form more cancer.

Interest in immunotherapies based on cancer vaccines and other methods of using a patient's own immune system to combat a tumor has increased in recent years and prompted attempts to develop such techniques as an additional treatment option for cancer patients. Such techniques would theoretically ensure elimination of all cancerous cells. Several types of cancer vaccines have been considered, including whole cell, defined tumor antigens, peptides and DNA vaccines. Except for whole cell vaccines, all other vaccines require a thorough understanding of the tumor antigen(s) involved. The non-whole cell vaccines, such as peptide or whole protein vaccines or antigen-specific DNA vaccines require a substantial knowledge of the expression of those tumor antigens, and their immunogenicity in cancer patients. Acquiring such data involves substantial investment in defining the tumor antigens. Most of these types of vaccines involve usage of one tumor antigen (e.g., telomerase antigen, prostatic acid phosphatase). However, effective anti-tumor immunity is complex, and multiple antigens and multiple epitopes are likely to be involved for efficacious clinical outcome. In almost all cancers except the ones induced by viruses, the tumor antigens are mostly self-proteins which have been tolerized during the development of the immune system, and hence it is difficult to induce an immune response against them. In the absence of a clear cut understanding of all tumor antigens involved in breaking self-tolerance and in the induction of clinically relevant immunity against cancer tissue, whole cell vaccines become good candidates for presenting a plethora of tumor antigens to the immune system, thereby hedging against tolerogenic epitopes.

The adaptive immune response to tumors alone is poor, mostly because the target antigens are self-proteins, except in cases of tumor induced by viruses. Since the immune system has evolved to recognize microbial/pathogenic organisms, it is possible that when self-antigens are presented by antigen presenting cells (APCs), there is normally no “danger” signal, and the APCs therefore provide tolerizing signals to avoid autoimmunity. In addition, many cancers suppress the major histocompatibility complex machinery, preventing cancer cells from presenting antigens that may be recognized as “non self” Several cancer vaccines have been tested in clinical trials, either alone or in conjunction with various adjuvants. Unfortunately, the clinical efficacy has thus far not been impressive.

The above-described deficiencies of today's systems are merely intended to provide an overview of some of the problems of conventional systems, and are not intended to be exhaustive. Other problems with the state of the art and corresponding benefits of some of the various non-limiting embodiments may become further apparent upon review of the following detailed description.

In view of the foregoing, it is desirable to provide an effective method of inducing a cancer patient's own immune system to identify and target cancerous cells.

### BRIEF SUMMARY OF THE INVENTION

Disclosed is a method for direct vaccination with a therapeutic vector. A vector expressing an immunogenic viral or other exogenous polypeptide is injected into a tumor. The vector enters the tumor cells in vivo and the tumor cells express an immunogenic marker, such as a polypeptide or other molecule, that extends outside the cell wall. The polypeptide stimulates an immune response in vivo to the tumor, which acts as an internally generated immuno-stimulant. A plasmid vector (such as pAc, pSI, pcDNA3.1 or any one of a large number of commercially available expression vectors) expressing this immunogenic polypeptide is injected into the tumor. The vector transforms the cancer cells in the tumor which then expresses the encoded polypeptide. The transformed cancer cells thus become in vivo/in situ vaccines against the tumor cells. This priming event then leads to further enhanced immune recognition of endogenous tumor associated or tumor specific antigens.

Once an immune response starts at a tumor site, due to the insertion of foreign genes, your immune system begins to recognize all the other things that are different about the tumor. At this point, the immune system begins to attack all the tumor cells, not just the ones that were originally altered to look like a virus or bacteria. These well trained immune cells then travel throughout the body and recognize all the cancer cells at distant sites and destroys every single one of them ridding the body of all tumors. And, just like getting vaccinated against anything else, if the cancer does come back, your immune system is already primed and ready to fight it.

There are many immunogenic viral or other exogenous markers which can significantly improve an immune response to cancer cells when these cancer cells have been transformed to express these proteins. The vaccines can be utilized to augment or enhance conventional anti-cancer treatments either as a primary or an adjuvant treatment.

The anticancer vector can be administered directly into tumor lesions using lipid reagents, needleless injectors, multi-needle administration patches, in vivo electroporation, J-tip, into palpable tissue, or visceral tumor lesions with the guidance of computed tomography (CT) or ultrasound. In using the direct administration of the DNA vector, there is no need to harvest tumor cells from the patient. Processing the cells, transformation and irradiation steps are not required so that expense is reduced and efficiency increased compared with the in vitro process of preparing the cancer vaccine ex vivo.

These and other objects and advantages of the present invention will become apparent from a reading of the attached specification and appended claims. There has thus been outlined, rather broadly, the more important features of the invention in order that the detailed description thereof that follows may be better understood, and in order that the present contribution to the art may be better appreciated. There are features of the invention that will be described hereinafter and which will form the subject matter of the claims appended hereto.

### DETAILED DESCRIPTION

The invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

The disclosed subject matter is described with reference to the drawings, wherein like reference numerals are used to refer to like elements throughout. In the following description, for purposes of explanation, numerous specific details are set forth in order to provide a thorough understanding of the various embodiments of the subject disclosure. It may be evident, however, that the disclosed subject matter may be practiced without these specific details. In other instances, well-known structures and devices are shown in block diagram form in order to facilitate describing the various embodiments herein. Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

Unless otherwise indicated, all numbers expressing quantities of ingredients, dimensions, reaction conditions and so forth used in the specification and claims are to be understood as being modified in all instances by the term “about”. The term “a” or “an” as used herein means “at least one” unless specified otherwise. In this specification and the claims, the use of the singular includes the plural unless specifically stated otherwise. In addition, use of “or” means “and/or” unless stated otherwise. Moreover, the use of the term “including”, as well as other forms, such as “includes” and “included”, is not limiting. Also, terms such as “element” or “component” encompass both elements and components comprising one unit and elements and components that comprise more than one unit unless specifically stated otherwise.

Various embodiments of the disclosure could also include permutations of the various elements recited in the claims as if each dependent claim was a multiple dependent claim incorporating the limitations of each of the preceding dependent claims as well as the independent claims. Such permutations are expressly within the scope of this disclosure.

Disclosed are therapeutic methods for use in stimulating anticancer immunity in cancer patients. Vectors such as plasmids are constructed to express immunogenic polypeptides such as viral proteins, immunogenic regions of viral proteins, or any other non-human immunogenic protein. These vectors are injected intratumorally, i.e. directly into a tumor. The vectors transform cancerous cells, causing them to express the immunogenic polypeptides. These peptides are then presented on the exterior of the cancerous cells, thereby generating an immune response.

The vector expressed polypeptides may be immunogenic proteins or immunogenic fragments of proteins commonly found in viruses that humans or other mammals are commonly vaccinated for, such as for example measles, mumps and/or rubella. As a result, the body's natural response, heightened by the vaccination, is utilized to attack the cancerous cells. In addition, the use of viral peptides also triggers the antiviral cytotoxic immunological responses, further increasing the immune systems response time, strength and efficacy. The use of a body's natural immunological response to treat cancer also avoids the undesirable side effects of other anticancer therapies.

Suitable polypeptides from the measles virus include: Hemagglutinin (H), Neurominidase (N), Nucleoprotein (Np), Fusion (F), Hemagglutinin noose epitope (HNE; aa 379-410), MeV (aa 89-165) (DAG1597), MeV Hemagglutinin Mosaic (DAG1598), MeV (aa 399-525) (DAG1599), MeV Fusion Mosaic protein (DAG1600), MeV Hemagglutinin Mosaic (aa 1-30, 115-150, 379-410) (DAG2298), MeV Fusion protein (aa 399-525) (DAG3566), MeV Hemagglutinin Mosaic (aa 106-114, 519-550) (DAG3567), MeV (aa 399-525) (DAG4038), MeV Hemagglutinin Fusion Protein (aa 399-525) (DAG4317), MeV Nucleocapsid (aa 89-165) (DAG4318), and Measles Active Nucleocapsid (DAG-P2799).

Suitable polypeptides from the Epstein Barr virus include: EBV Mosaic EBNA1 protein [GST] (DAG1577), EBV NA1 (aa 1-90, 408-498) [GST] (DAG2362), EBV gp350/220, EBV P18 Mosaic protein (DAG1582), EBV BFRF3 [GST] (DAG1848), EBV BMRF1 [GST] (DAG1849), EBV EBNA1 [GST] (DAG1850), EBV Early Antigen (aa 306-390) (DAG2003), EBV Glycoprotein H(DI-II), gL, Gp 42 (aa 25-137)(Ectodomain) [His] (DAG2016), EBV Glycoprotein H(DI-III), and gL, Gp 42 (aa 31-223)(Ectodomain) [His] (DAG2017).

Suitable polypeptides from the Varicella Zoster virus include: VZV Glycoprotein E [GST] (DAG518), VZV Glycoprotein (DAG3110), VZV Glycoprotein E (aa 48-135) [GST] (DAG3633), VZV Envelope glycoprotein E (aa 48-135) (DAG-P2161), VZV Active Glycoprotein E (aa 48-135) (DAG-P2598), and VZV ORF9 (DAG-P2622).

Suitable polypeptides from the Rubella virus include: RuV E1 glycoprotein, RuV Envelope Protein 1 (DAG3134), RuV Nucleocapsid (full length) (DAG-P2986), RuV Capsid (aa 1-123) (DAG1979), RuV E1 protein (aa 157-276) [His] (DAG2000), RuV E2 Protein (aa 31-105) [His] (DAG2002), RuV E1 Mosaic (aa 157-176) (DAG3583), RuV Capsid C [GST] (DAG4369), RuV E1 protein (E1) [GST] (DAG472), RuV E1 Mosaic [His] (DAG488), RuV Capsid (DAG494), and RuV E1 Mosaic (DAG507).

Other suitable viral poly peptides include: Mumps Fusion (F) and Hemagglutinin/neurominadase (HN), Vaccinia virus H3L (an intracellular mature virion envelope protein) and virus B18R protein, Lymphocytic choriomeningitis virus (LCMV) cytotoxic T lymphocyte (CTL) epitope (NP118-126), Human Cytomegalovirus (HCMV) fusion protein gB, and two highly conserved anti-parallel (3-strands on the pre-fusion viral F protein of the Human respiratory syncytial virus (HRSV).

The immune response generated by these polypeptides will initially be directed against the viral antigen expressed on the surface of the tumor. However, the immune system cells, e.g. T cells, dendritic cells, NK cells and others, that infiltrate will begin to recognize the many different tumor associated and tumor specific antigens that make the cancer cell different. This is known as epitope spreading and is well documented. Epitope spreading allows these primed immune cells to travel throughout the body and recognize and destroy distant metastatic cancer cells that do not express the viral antigens thus eliminating all cancer cells and tumors throughout the body.

One advantage of using viral antigens over bacterial or endogenous (even tumor specific or associated antigens) is that the immune response to viruses is predominantly a cytotoxic T lymphocyte (CTL) response comprised of CD8+ and CD4+ and other effector cells which are the cells that are most effective at inducing cytolysis of tumor cells. This is superior to other regimens such as bacterial antigen expressing systems which primarily induce a humoral or B cell response. While the hum oral response may be somewhat effective, it is hypothesized that an anti-tumor response that is predominantly CTL mediated will be significantly more effective.

In some embodiments, viral fusion proteins that add a transmembrane tail may be incorporated into the expression vector. Those skilled in the art will appreciate that these viral fusion proteins may be anchored to the surface of the cancer cell and the therefore will be identified by memory T cells as a result of a previous vaccination. These viral fusion proteins may also migrate to a cancerous cell's membrane without the assistance of the major histocompatibility complex (MHC) which is compromised in some cancer cell types.

In other embodiments, the viral polypeptide incorporated into the expression vector is a viral protein known to be found on the membrane of an infected cell. Such polypeptides may use a host transport mechanisms that the virus is known to employ as a infection strategy.

In other embodiments, the immunogenic protein that is incorporated in the expression vector may be of bacterial origin. This may include modified immunogens such as tetanus toxoid. The obvious utility of this being that many people are already vaccinated against it.

In other embodiments the polypeptide is an antigenic portion of an MHC molecule from a non-human mammal. It is known that disparate MHC matching causes a robust immune response and therefore these polypeptides will elicit a robust immune response.

In other embodiments the polypeptide is from a non-mammalian animal source that is known to induce a potent immune response. These may be derived from species known to induce a cytotoxic adaptive immune response.

In other embodiments the polypeptide is from a non-animal source that is known to induce a potent immune response. These may be derived from plant or fungal species known to induce a cytotoxic adaptive immune response.

The expression of the immunogenic polypeptide, whether of viral origin or another source as described above, in tumor cells greatly increases tumor cell immunogenicity, which in turn promotes activation of both innate and adaptive antitumor immunity. A clinically meaningful response against tumor cells, such as tumor regression/prevention of recurrence results from in vivo priming and activation of the immune system.

The disclosed anticancer vector can also be used in conjunction with monoclonal antibodies to checkpoint inhibitory molecules such as CTLA-4, PD-1, PD-L1, PD-L2, LAG3, TIM3, TIGIT; antibodies to costimulatory molecules such as CD40, OX40; antibodies capable of regulating T regs such as anti-GITR and pan anti-BCL-2; or cytokines such as IL-2, TNF-α, IFN-γ, IFN-β, and TLR agonists administered in normal fashion for as the currently are for anti-cancer therapy.

The anticancer vector coding for the antigenic polypeptide can also comprise additional nucleic acid sequences that express immunologic molecules such as cytokines IL-2, IL-12, IL-18, CD-40L, CTLA-4 ligand and MHC genes. In this embodiment both the antigenic polypeptide and the costimulatory molecule are engineered into the same plasmid. Thus, a single vector plasmid will lead to expression of the antigenic polypeptide which will induce a priming response and the costimulatory molecule will overcome the inhibition that tumor cells exhibit.

Anti-Cancer Vectors. A wide variety of vectors, for example plasmids, may be utilized to deliver viral polypeptide genes into cancerous cells in accordance with principles of the invention. The vectors may include promoter and enhancer regions to increase expression of the exogenous polypeptides.

Delivery of Anticancer Vector into a Solid Tumor In Vivo. The anticancer vectors are injected directly into a tumor where they enter cancerous cells.

Whereas, the present invention has been described in relation to the drawings attached hereto, other and further modifications, apart from those shown or suggested herein, may be made within the spirit and scope of this invention. Those skilled in the art will appreciate that the conception, upon which this disclosure is based, may readily be utilized as a basis for the designing of other structures, methods and systems for carrying out the several purposes of the present invention. Descriptions of the embodiments shown in the drawings should not be construed as limiting or defining the ordinary and plain meanings of the terms of the claims unless such is explicitly indicated. The claims should be regarded as including such equivalent constructions insofar as they do not depart from the spirit and scope of the present invention.

1. A method of treating cancer in a mammal comprising transfecting tumors in vivo and in situ with a vector (plasmid or viral) that expresses an exogenous immunogenic polypeptide of viral, fungal, bacterial, non-human mammal or non-animal plant origin.

2. The method of treating cancer in a mammal of claim 1, wherein the immunogenic polypeptide is one or more polypeptides derived from the measles virus selected from the group consisting of Hemagglutinin (H), Neurominidase (N), Nucleoprotein (Np), Fusion (F), Hemagglutinin noose epitope (HNE; aa 379-410), MeV (aa 89-165) (DAG1597), MeV Hemagglutinin Mosaic (DAG1598), MeV (aa 399-525) (DAG1599), MeV Fusion Mosaic protein (DAG1600), MeV Hemagglutinin Mosaic (aa 1-30, 115-150, 379-410) (DAG2298), MeV Fusion protein (aa 399-525) (DAG3566), MeV Hemagglutinin Mosaic (aa 106-114, 519-550) (DAG3567), MeV (aa 399-525) (DAG4038), MeV Hemagglutinin Fusion Protein (aa 399-525) (DAG4317), MeV Nucleocapsid (aa 89-165) (DAG4318), and Measles Active Nucleocapsid (DAG-P2799).

3. The method of treating cancer in a mammal of claim 1, wherein the immunogenic polypeptide is one or more polypeptides derived from the Epstein Barr virus selected from the group consisting of EBV Mosaic EBNA1 protein [GST] (DAG1577), EBV NA1 (aa 1-90, 408-498) [GST] (DAG2362), EBV gp350/220, EBV P18 Mosaic protein (DAG1582), EBV BFRF3 [GST] (DAG1848), EBV BMRF1 [GST] (DAG1849), EBV EBNA1 [GST] (DAG1850), EBV Early Antigen (aa 306-390) (DAG2003), EBV Glycoprotein H(DI-II), gL, Gp 42 (aa 25-137)(Ectodomain) [His] (DAG2016), EBV Glycoprotein H(DI-III), and gL, Gp 42 (aa 31-223)(Ectodomain) [His] (DAG2017).

4. A method of treating cancer in a mammal comprising transfecting tumor cells in vivo using a vector including and expressing an exogenous immunogenic polypeptide of viral, fungal, non-human mammal or non-animal plant origin that also includes an immunostimulatory moiety such that both the immunogenic polypeptide and the immunostimulatory entity are co-expressed at the same time in the same transfected cells.
